(19) United States
(12) Patent Application Publication
(10) Pub. No.: US 2024/0293485 A1
(43) Pub. Date: Sep. 5, 2024

(54) COMPOSITIONS AND METHODS USING A COMBINATION OF AT LEAST ONE FIBER AND AT LEAST ONE PROBIOTIC TO IMPROVE MICROBIOME RESILIENCE

(71) Applicant: SOCIETE DES PRODUITS NESTLE S.A., Vevey (CH)

(72) Inventors: SAMI DAMAK, Lausanne (CH); MARC GARCIA-GARCERA, Ecublens (CH); BERNARD BERGER, Maracon (CH); FABIO MAINARDI, Lausanne (CH); CLARA LUCIA GARCIA-RODENAS, Forel (CH); SHAILLAY KUMAR DOGRA, Epalinges (CH)

(21) Appl. No.: 18/572,429

(22) PCT Filed: Jun. 21, 2022
(86) PCT No.: PCT/EP2022/066799

§ 371 Date: Dec. 20, 2023

Related U.S. Application Data
(60) Provisional application No. 63/213,280, filed on Jun. 22, 2021.

(51) Int. Cl. A61K 35/747; A61K 31/702; A61K 31/717; A61K 31/732; A61K 31/736; A61K 35/00; A61K 35/745; A61K 36/42; A61K 36/55; A61K 36/736; A61P 1/14

(52) U.S. Cl. CPC A61K 35/747; A61K 31/702; A61K 31/717; A61K 31/732; A61K 31/736; A61K 35/745; A61K 36/42; A61K 36/55; A61K 36/736; A61P 1/14; A61K 2035/115

(57) ABSTRACT

A composition contains a combination of at least one fiber and at least one probiotic and is formulated for administration to a subject such that the combination of at least one fiber and at least one probiotic enhance resilience of the microbiome in the subject. Preferably the subject is experiencing a microbiome stressor, will experience a microbiome stressor and/or has recently experienced a microbiome stressor, such as one or more of a dietary stressor, antibiotic, other medications, infections, intense exercise, stress, alcohol, travel and combinations thereof; more preferably at least a dietary stressor; most preferably at least a high fat diet, such as a Western high fat diet or a ketogenic diet. The method can achieve at least one result that is (i) prevention or attenuation of perturbation of microbiota; (ii) recovery after perturbation; and (iii) normalization of one or more of stool frequency, intestinal transit, constipation, gut permeability, endotoxemia or gut barrier function.

### BACKGROUND

The present disclosure generally relates to methods to improve microbiome resilience. The present disclosure further relates to compositions useful in these methods and also relates to methods of making such compositions.

The human gut microbiome is an ecosystem of trillions of bacteria. Throughout life, the gut microbiome is challenged by one or more of unhealthy diet, antibiotics, other medications, infections, intense exercise, or alcohol. The ability of the microbiome to resist those challenges or quickly and fully recover from the perturbation is “microbiome resilience” and likely contributes to maintaining health. Reduced microbiome resilience may lead to dysbiosis with negative impact on health.

### SUMMARY

There is mounting evidence that the gut microbiome plays a key role in health and disease. However, the resilience of the microbiota after a high fat diet stress has not been addressed previously. The clinical study disclosed herein demonstrates that intake of a combination of a fiber blend and a probiotic mixture improve microbiome resilience, intestinal transit, and gut barrier in subjects undergoing a challenge of a diet with high fat and low fiber. Accordingly, the present disclosure generally relates to a novel improvement of microbiome resilience by a combination of a fiber blend and a probiotic mixture to thereby improve gastrointestinal health.

Benefits from this improvement of microbiota resilience may include, for example, prevention or attenuation of perturbation of microbiota; management of dysbiosis; recovery after perturbation; and normalization of one or more of stool frequency, intestinal transit, constipation, gut permeability, endotoxemia or gut barrier function (e.g., against endotoxins).

Additional features and advantages are described herein and will be apparent from the following Figures and Detailed Description.

### BRIEF DESCRIPTION OF DRAWINGS

FIG. 1 is a schematic diagram generally illustrating microbiota resilience as defined herein.

FIG. 2 is a schematic diagram showing the clinical trial design in the experimental example disclosed herein.

FIG. 3A is a table showing the formulation of the fiber product used in the experimental example disclosed herein.

FIG. 3B is a table showing the formulation of the probiotic product used in the experimental example disclosed herein.

FIG. 4 is a graph showing an effect of the intervention on the prevalence of the microbiota members overtime.

FIG. 5 is a graph showing the effect of the intervention and the high fat diet challenge on the alpha-diversity.

FIG. 6 is a graph showing results from the experimental example disclosed herein, demonstrating the microbiota is less perturbed by high fat diet when the nutritional intervention is given.

FIG. 7 is a graph showing the changes in the community structure of the microbiota.

FIGS. 8A-8C include two graphs and a table showing results regarding resistance index from the experimental example disclosed herein; Resistance=1/dmax; dmax=maximum Aitchison distance during the challenge, with respect to baseline (day=0).

FIG. 9A is a table showing results regarding recovery index from the experimental example disclosed herein; Recovery=time to reach the minimum Aitchison distance after the peak; Average recovery is by group and by taxonomical level.

FIG. 9B is a graph showing the results regarding recovery index at family level.

FIG. 10 is a graph showing the differences in metabolic composition with respect to a baseline.

FIG. 11 is a graph showing results regarding effects on the host from the experimental example disclosed herein, demonstrating the nutritional intervention normalizes stool frequency during the challenge.

FIGS. 12A and 12B are graphs showing results regarding effects on the host from the experimental example disclosed herein, demonstrating increased high fat diet-induced antibodies against circulating endotoxin levels are prevented by the nutritional intervention.

### DETAILED DESCRIPTION

### Definitions

Some definitions are provided hereafter. Nevertheless, definitions may be located in the “Embodiments” section below, and the above header “Definitions” does not mean that such disclosures in the “Embodiments” section are not definitions.

All percentages expressed herein are by weight of the total weight of the composition unless expressed otherwise. As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number. All numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used in this disclosure and the appended claims, the singular forms “a,” “an” and “the” include plural referents unless the context clearly dictates otherwise. Thus, for example, reference to “a vitamin” or “the vitamin” encompass both an embodiment having a single vitamin and an embodiment having two or more vitamins.

The words “comprise,” “comprises” and “comprising” are to be interpreted inclusively rather than exclusively. Likewise, the terms “include,” “including” and “or” should all be construed to be inclusive, unless such a construction is clearly prohibited from the context. Nevertheless, the compositions disclosed herein may lack any element that is not specifically disclosed herein. Thus, a disclosure of an embodiment using the term “comprising” includes a disclosure of embodiments “consisting essentially of” and “consisting of” the components identified.

The terms “at least one of” and “and/or” used in the respective context of “at least one of X or Y” and “X and/or Y” should be interpreted as “X,” or “Y,” or “X and Y.” For example, “at least one of resistance or recovery” and “resistance and/or recovery” should be interpreted as “resistance,” or “recovery,” or “both resistance and recovery.”

Where used herein, the terms “example” and “such as,” particularly when followed by a listing of terms, are merely exemplary and illustrative and should not be deemed to be exclusive or comprehensive. As used herein, a condition “associated with” or “linked with” another condition means the conditions occur concurrently, preferably means that the conditions are caused by the same underlying condition, and most preferably means that one of the identified conditions is caused by the other identified condition.

“Prevention” includes reduction of risk, incidence and/or severity of a condition or disorder. The terms “treatment” and “treat” include both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of patients at risk of contracting a disease or suspected to have contracted a disease, as well as patients who are ill or have been diagnosed as suffering from a disease or medical condition. The terms “treatment” and “treat” do not necessarily imply that a subject is treated until total recovery. The terms “treatment” and “treat” also refer to the maintenance and/or promotion of health in an individual not suffering from a disease but who may be susceptible to the development of an unhealthy condition. The terms “treatment” and “treat” are also intended to include the potentiation or otherwise enhancement of one or more primary prophylactic or therapeutic measures. As non-limiting examples, a treatment can be performed by a patient, a caregiver, a doctor, a nurse, or another healthcare professional.

As used herein, a prophylactically or therapeutically “effective amount” is an amount that prevents a deficiency, treats a disease or medical condition in an individual, or, more generally, reduces symptoms, manages progression of the disease, or provides a nutritional, physiological, or medical benefit to the individual. The relative terms “promote,” “improve,” “increase,” “enhance” and like terms refer to resilience of the microbiome of the subject (i.e., resistance to challenge and/or recovery from challenge), after administration of the composition disclosed herein (which comprises a fiber and a probiotic), relative to the resilience of the microbiome of the subject obtained by administration of a composition lacking the fiber and/or the probiotic but otherwise identically formulated.

As used herein, the terms “food,” “food product” and “food composition” mean a product or composition that is intended for oral ingestion by a human or other mammal and comprises at least one nutrient for the human or other mammal.

“Nutritional compositions” and “nutritional products,” as used herein, include any number of food ingredients and possibly optional additional ingredients based on a functional need in the product and in full compliance with all applicable regulations. The optional ingredients may include, but are not limited to, conventional food additives, for example one or more, acidulants, additional thickeners, buffers or agents for pH adjustment, chelating agents, colorants, emulsifies, excipient, flavor agent, mineral, osmotic agents, a pharmaceutically acceptable carrier, preservatives, stabilizers, sugar, sweeteners, texturizers, and/or vitamins. The optional ingredients can be added in any suitable amount.

“Probiotic” means microbial cell preparations or components of microbial cells with a beneficial effect on the health or well-being of the host. (Salminen S, Ouwehand A. Benno Y. et al “Probiotics: how should they be defined” Trends Food Sci. Technol. 1999:10 107-10).

The term “unit dosage form,” as used herein, refers to physically discrete units suitable as unitary dosages for human and animal subjects, each unit containing a predetermined quantity of the composition disclosed herein in an amount sufficient to produce the desired effect, in association with a pharmaceutically acceptable diluent, carrier or vehicle. The specifications for the unit dosage form depend on the particular compounds employed, the effect to be achieved, and the pharmacodynamics associated with each compound in the host.

A “subject” or “individual” is a mammal, preferably a human.

### Embodiments

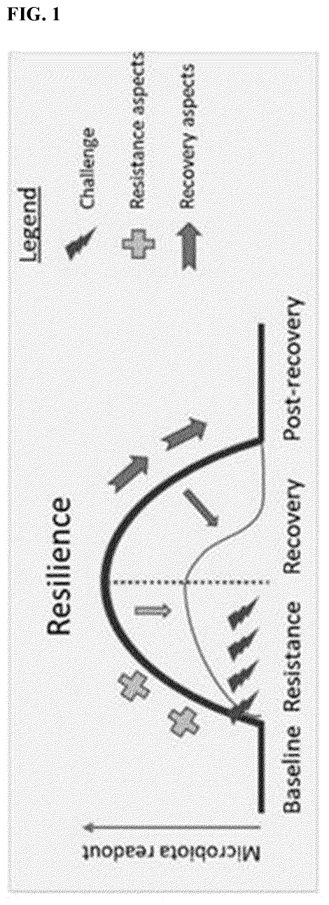

As shown in FIG. 1, microbiota resilience is defined as the capacity to resist or fully and quickly recover from challenge or perturbation. Accordingly, an aspect of the present disclosure is a composition comprising a combination of at least one fiber and at least one probiotic and formulated for administration to a subject such that the combination enhances resilience of the microbiome in the subject.

Another aspect is a method to enhance resilience of the microbiome in a subject (e.g., a subject in need thereof), the method comprising administering, to the subject, a composition comprising a combination of at least one fiber and at least one probiotic (e.g., in a therapeutically effective or a prophylactically effective amount). In an embodiment, the subject is experiencing a microbiome stressor and/or has recently experienced a microbiome stressor (e.g., less than one year ago, preferably less than one month ago, more preferably less than one week ago, most preferably one day ago or less).

A related embodiment is a method of treating, preventing, reducing an incidence of, and/or reducing a severity of condition associated with a microbiome stressor in a subject who is experiencing the microbiome stressor, has recently experienced the microbiome stressor (e.g., within the most recent month or within the most recent week), and/or will experience the microbiome stressor in the near future (e.g., within the upcoming month or within the upcoming week), the method comprising administering, to the subject, a composition comprising a combination of at least one fiber and at least one probiotic (e.g., in a therapeutically effective or a prophylactically effective amount).

In a preferred embodiment, the stressor is a dietary stressor, such as a high fat diet, for example a Western diet or a ketogenic diet, or a low carbohydrate diet. As used herein, a high fat diet is a daily caloric intake in which greater than 35% of the daily caloric intake is from dietary fat, such as at least about 40% of the daily caloric intake from dietary fat, at least about 45% of the daily caloric intake from dietary fat, at least about 50% of the daily caloric intake from dietary fat, at least about 55% of the daily caloric intake from dietary fat, or at least about 60% of the daily caloric intake from dietary fat.

A Western diet is characterized by its highly processed and refined foods; high contents of sugars, salt, and fat; protein from red meat; and low content in fibers. “Low fiber” is considered as a diet with less than 15 g of fibers per 2000 calories per day.

As used herein, a low carbohydrate diet has no greater than about 15% of the daily caloric intake from carbohydrates, such as no greater than about 10% of the daily caloric intake from carbohydrates or no greater than about 5% of the daily caloric intake from carbohydrates.

In some embodiments, the subject has been consuming a high fat diet (e.g., a Western diet, or a ketogenic diet) or a low carbohydrate diet for at least one day (e.g., at least one week or at least one month) prior to a first administration of the combination of at least one fiber and at least one probiotic, with optional subsequent administrations of the combination of at least one fiber and at least one probiotic (e.g., daily administration over a time period of at least one week or at least one month).

Additionally or alternatively, the stressor can comprise one or more of: antibiotic; other medications; infections; intense exercise; stress; alcohol; travel; parenteral feeding; enteral feeding; short bowel syndrome; gut inflammation; chemotherapy; colon cancer; diarrhea; proton pump inhibitors; gluten-free diet; diet free of fermentable oligo-, di-, mono-saccharides and polyols (FODMAPs); or combinations thereof.

In some embodiments, the subject consumes one of the compositions disclosed herein on a daily basis, for example each day for at least one week prior to the stressor or even at least one month prior to the stressor.

Yet another aspect is a unit dosage form of a composition comprising a combination of at least one fiber and at least one probiotic, the unit dosage form comprising an amount of the combination (of at least one fiber and at least one probiotic) effective to enhance resilience of the microbiome in a subject to whom the unit dosage form is administered.

A further aspect is a method of making a composition to enhance resilience of the microbiome of a subject to whom the composition is administered, the method comprising adding at least one fiber to at least one probiotic and preferably to at least one additional component.

Another aspect is a method of achieving at least one result selected from the group consisting of (i) prevention or attenuation of perturbation of microbiota; (ii) recovery after perturbation; and (iii) normalization of one or more of stool frequency, intestinal transit, constipation, gut permeability, endotoxemia, or gut barrier function, the method comprising enhancing resilience of the microbiome in a subject (e.g., a subject in need thereof) by administering, to the subject, a composition comprising a combination of at least one fiber and at least one probiotic (e.g., in an effective amount).

Yet another aspect is a method of improving gastrointestinal health, the method comprising enhancing resilience of the microbiome in a subject (e.g., a subject in need thereof) by administering, to the subject, a composition comprising a combination of at least one fiber and at least one probiotic (e.g., in an effective amount).

In the embodiments disclosed herein, each of the at least one fiber is edible, meaning that all of the components of the fibers are safe and suitable for consumption by humans and/or animals. The at least one fiber comprises insoluble fiber and/or soluble fiber, preferably a blend of insoluble fiber and soluble fiber. In some embodiments, the at least one fiber can be selected from the group consisting of xylooligosaccharides, flax seed, partially hydrolyzed guar gum (PHGG), glucomannan, cellulose, prune powder, pectin such as apple peel pectin, and mixtures thereof. In some embodiments, the at least one fiber is at least two fibers, such as two, three, four, five, six or seven fibers and optionally more. Optionally one or more of Luo Han Guo fruit powder, xylitol or magnesium (e.g., magnesium citrate) can be included with the at least one fiber.

In some embodiments, the at least one probiotic can be selected from the group consisting of Lactobacillus acidophilus, Bifidobacterium lactis, Lactobacillus rhamnosus, Bifidobacterium longum, Lactobacillus plantarum, Bifidobacterium bifidum, Lactobacillus gasseri, and mixtures thereof. In some particular embodiments, the at least one probiotic can be a strain selected from the group consisting of Lactobacillus acidophilus La-14, Bifidobacterium lactis BI-04, Lactobacillus rhamnosus GG, Bifidobacterium longum BL-05, Lactobacillus plantarum Lp-115, Bifidobacterium bifidum Bb-06, Lactobacillus gasseri Lg-36, and mixtures thereof. In some embodiments, the at least one probiotic is at least two probiotic strains, such as two, three, four, five, six or seven probiotic strains and optionally more.

The at least one fiber may be administered to the individual as a total daily dose of about 5-40 g, preferably about 15-25 g. The at least one fiber may be administered in a composition comprising between about 300-1000 mg total fiber/g of dry composition.

The at least one probiotic may be administered to the individual as a daily dose of 1×10³ to 1×10¹², preferably 1×10⁷ to 1×10¹¹ cfu (cfu=colony forming unit). The at least one probiotic may be administered in a composition comprising between 1×10³ to 1×10¹² cfu/g of dry composition. The at least one probiotic may be alive, fragmented, or in the form of fermentation products (e.g., supernatant) or metabolites, or a mixture of any or all of these states.

The combination of at least one fiber and at least one probiotic is preferably orally administered in a composition such as a food composition.

The subject to whom the combination of at least one fiber and at least one probiotic is administered can be selected from the group consisting of a human infant, a human child, a human adolescent, a human adult and an elderly human.

The combination of at least one fiber and at least one probiotic can be administered to the individual by at least one route selected from the group consisting of oral, topical, enteral and parenteral. For example, the combination of at least one fiber and at least one probiotic can be administered in a composition selected from the group consisting of a nutritionally complete product, a drink, a dietary supplement, a meal replacement, a food additive, a supplement to a food product, a powder for dissolution, an enteral nutrition product, an infant formula, a capsule, and combinations thereof.

Optionally the combination of at least one fiber and at least one probiotic is administered in a composition further comprising at least one component selected from the group consisting of an amino acid, a protein, a nucleotide, a fish oil, a non-marine source of omega-3 fatty acids, a phytonutrient, an antioxidant, and mixtures thereof.

The composition may be a food product, an animal food product, or a pharmaceutical composition. For example, the product may be a nutritional composition, a nutraceutical, a drink, a food additive or a medicament. A food additive or a medicament may be in the form of tablets, capsules, pastilles, a liquid, or a powder in a sachet, for example.

In some embodiments, the at least one probiotic is concurrently administered in a composition separate from the at least one fiber, for example in separate compositions administered to the same individual within one hour of each other, preferably within thirty minutes of each other, more preferably within ten minutes of each other, most preferably within one minute of each other.

The composition comprising the combination of at least one fiber and at least one probiotic is preferably selected from the group consisting of milk powder based products; instant drinks; ready-to-drink formulations; nutritional powders; nutritional liquids; milk-based products, in particular yoghurts or ice cream; cereal products; beverages; water; coffee; cappuccino; malt drinks; chocolate flavoured drinks; culinary products; soups; tablets; and/or syrups.

The composition may optionally comprise any milk obtainable from animal or plant sources, such as one or more of cow's milk, human milk, sheep milk, goat milk, horse milk, camel milk, rice milk or soy milk. Additionally or alternatively, milk-derived protein fractions or colostrum may be used.

The composition comprising the combination of at least one fiber and at least one probiotic may further contain protective hydrocolloids (such as gums, proteins, modified starches), binders, film forming agents, encapsulating agents/materials, wall/shell materials, matrix compounds, coatings, emulsifiers, surface active agents, solubilizing agents (oils, fats, waxes, lecithins etc.), adsorbents, carriers, fillers, co-compounds, dispersing agents, wetting agents, processing aids (solvents), flowing agents, taste masking agents, weighting agents, jellifying agents, gel forming agents, antioxidants and antimicrobials.

The composition comprising the combination of at least one fiber and at least one probiotic may also contain conventional pharmaceutical additives and adjuvants, excipients and diluents, including, but not limited to, water, gelatine of any origin, vegetable gums, ligninsulfonate, talc, sugars, starch, gum arabic, vegetable oils, polyalkylene glycols, flavouring agents, preservatives, stabilizers, emulsifying agents, buffers, lubricants, colorants, wetting agents, fillers, and the like. Further, the composition may contain an organic or inorganic carrier material suitable for oral or enteral administration as well as vitamins, minerals trace elements and other micronutrients in accordance with the recommendations of Government bodies such as the USRDA.

The composition comprising the combination of at least one fiber and at least one probiotic may optionally contain one or more amino acids, a protein source, a carbohydrate source and/or a lipid source, particularly in embodiments of the composition that are a food product.

Any suitable dietary protein may be used, for example animal proteins (such as milk proteins, meat proteins and egg proteins); vegetable proteins (such as soy protein, wheat protein, rice protein, and pea protein); mixtures of free amino acids; or combinations thereof. Milk proteins such as casein and whey, and soy proteins are particularly preferred.

The composition comprising the combination of at least one fiber and at least one probiotic may be administered to humans or animals, in particular companion animals, pets or livestock. It has beneficial effects for any age group. Preferably, the composition is formulated for administration to infants, juveniles, adults or elderly. In some embodiments, the composition can be administered to mothers during pregnancy and lactation to treat the infant.

The composition comprising the combination of at least one fiber and at least one probiotic can be administered at least one day per week, preferably at least two days per week, more preferably at least three or four days per week (e.g., every other day), most preferably at least five days per week, six days per week, or seven days per week. The time period of administration can be at least one week, preferably at least one month, more preferably at least two months, most preferably at least three months, for example at least four months. In an embodiment, dosing is at least daily; for example, a subject may receive one or more doses daily. In some embodiments, the administration continues for the remaining life of the individual. In other embodiments, the administration occurs until no detectable symptoms of the medical condition remain. In specific embodiments, the administration occurs until a detectable improvement of at least one symptom occurs and, in further cases, continues to remain ameliorated.

### Example

The following non-limiting example generally illustrates the concepts underlying the embodiments disclosed herein.

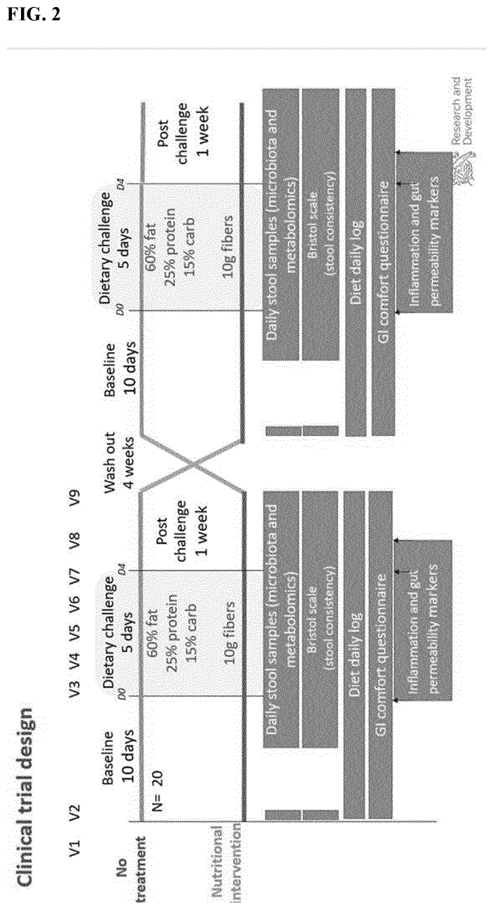

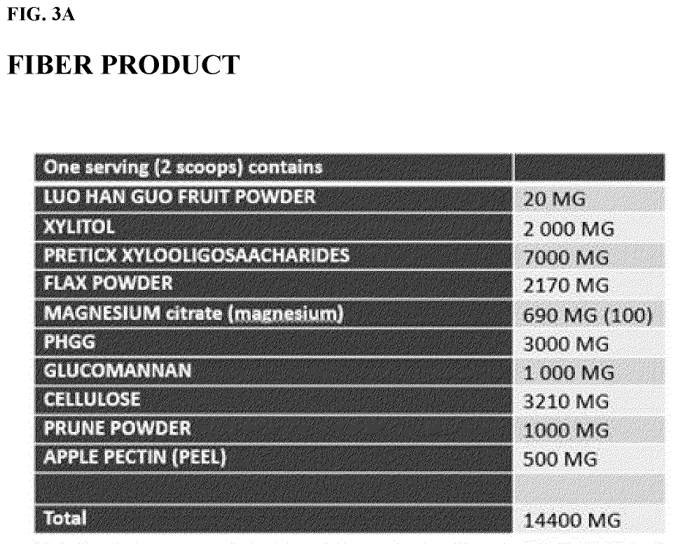

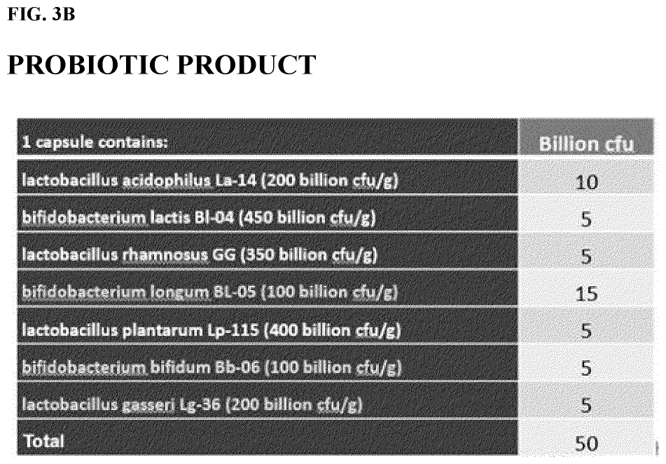

FIG. 2 shows the clinical trial design, which used a fiber product containing a diverse blend of fibers to improve overall microbiome diversity and a probiotic product to replenish down-regulated bacteria, diminish inflammation, and improve gut barrier. FIG. 3A shows the formulation of the fiber product, and FIG. 3B shows the formulation of the probiotic product.

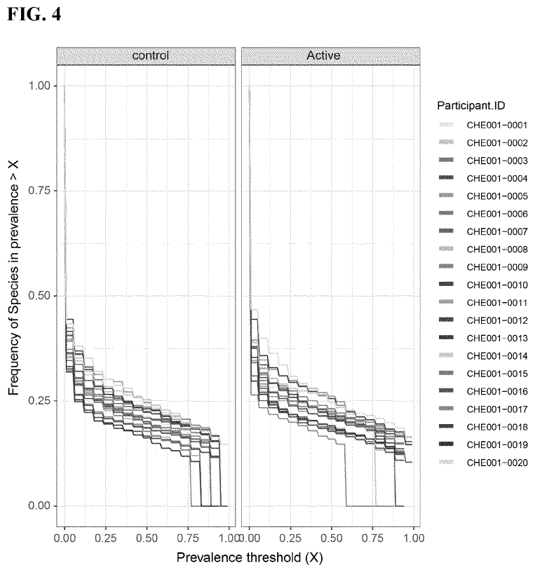

The results are shown in FIGS. 4 to 12. Specifically, FIG. 4 shows an effect of the intervention on the prevalence of the microbiota members overtime. The intervention increases the overall prevalence of microbiota members throughout the experiment; this increase in prevalence highlights a higher stability of the microbiota after a high fat diet challenge compared to the control.

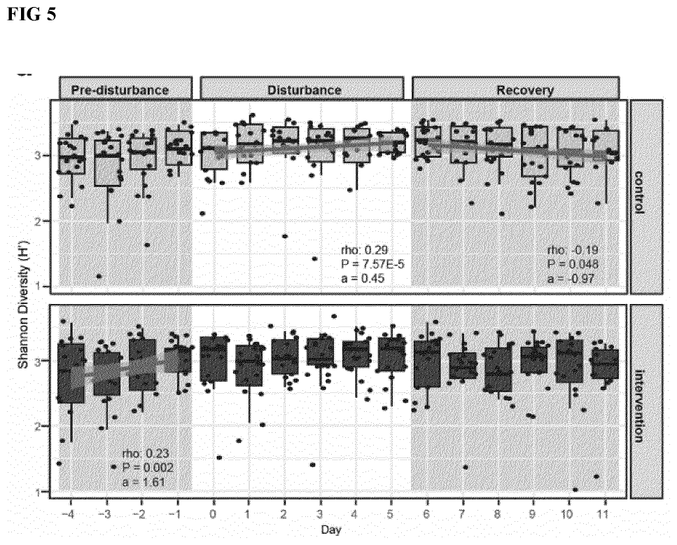

FIG. 5 is showing an effect of the intervention on alpha diversity (defined as the diversity of microorganisms that are present in an ecosystem). The intervention significantly increases the alpha-diversity of the ecosystem before the high fat diet challenge. Moreover, the intervention maintains stable the alpha diversity during the high fat diet challenge.

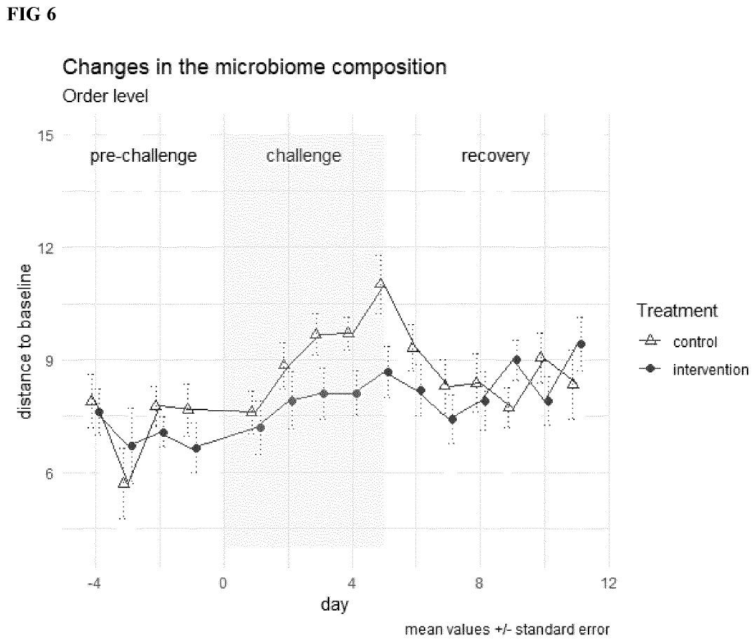

FIG. 6 demonstrates the microbiota is less perturbed by high fat diet when the nutritional intervention is given.

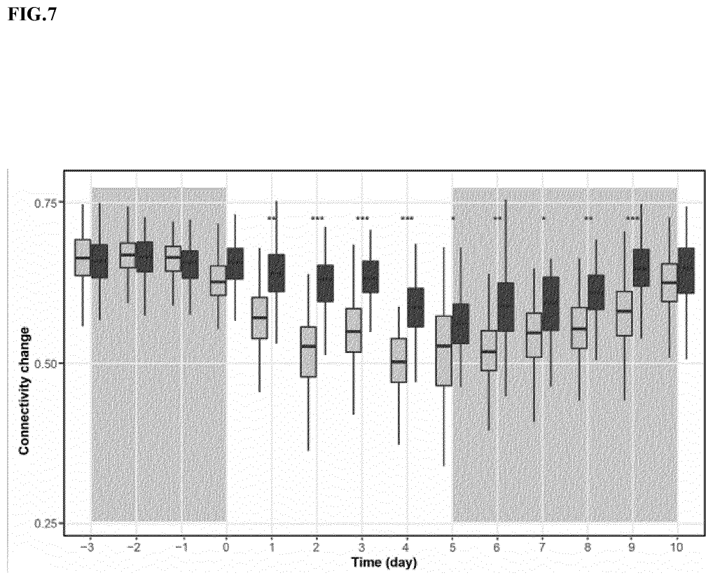

FIG. 7 shows the changes in the community structure of the microbiota (defined as the number and specificity of interactions between the members of the microbiota). The intervention maintains the number and specificity of interactions during the high fat diet challenge.

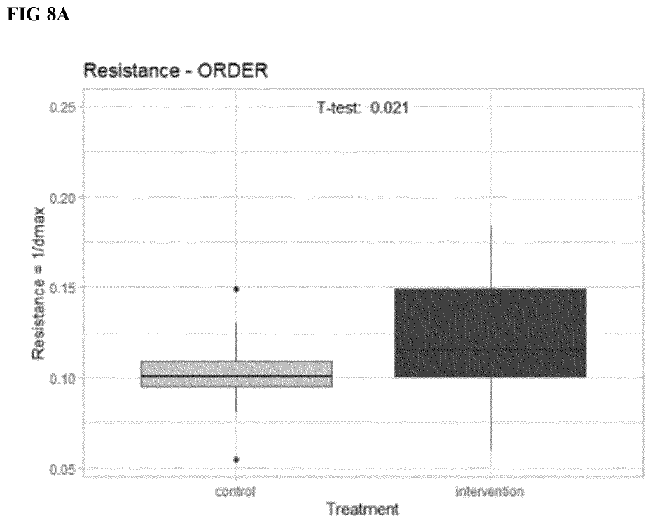

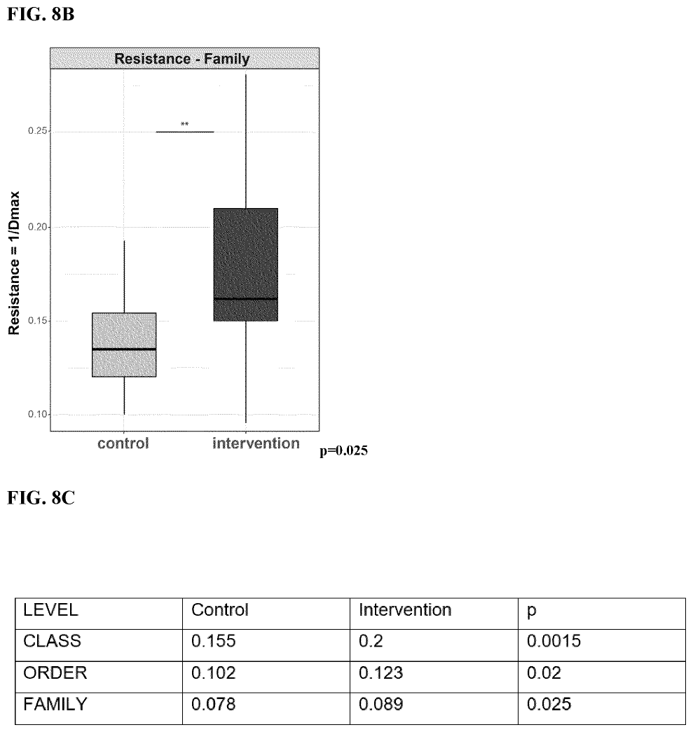

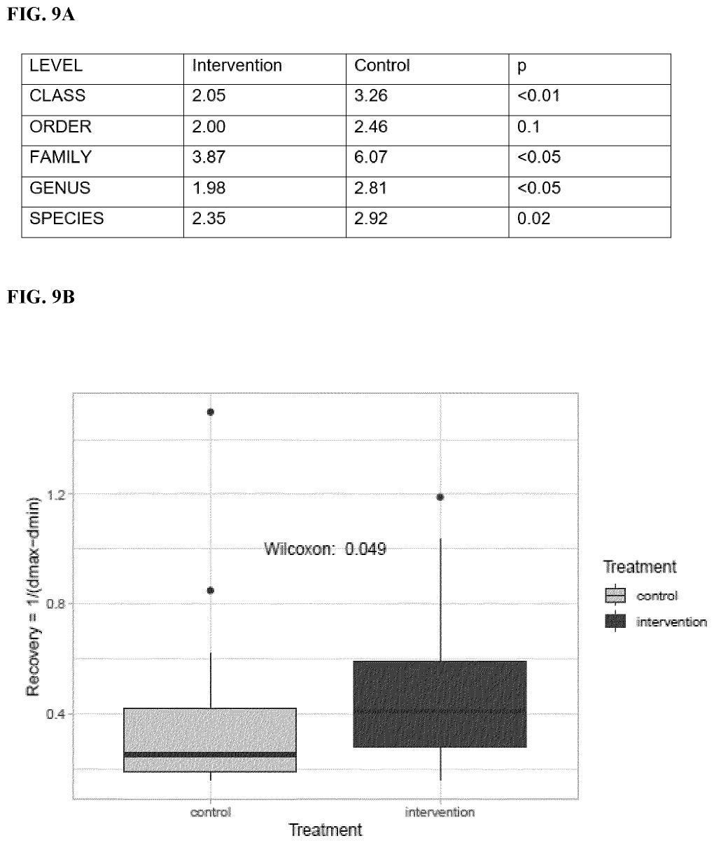

FIGS. 8A-8C show results regarding resistance index, and FIG. 9 shows results regarding recovery index. Intervention decreases the recovery time after the high fat diet challenge.

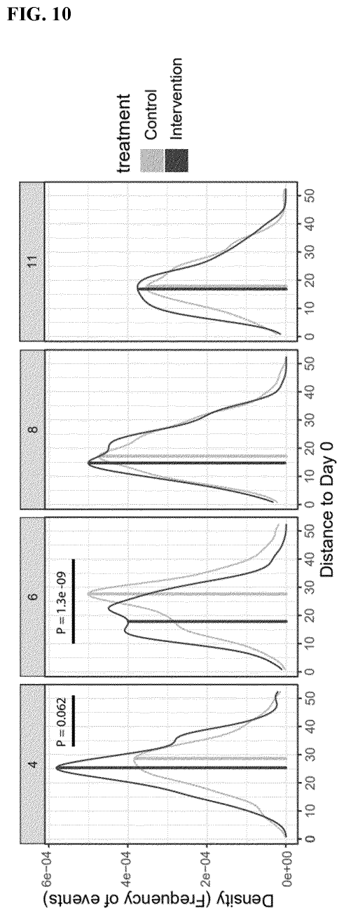

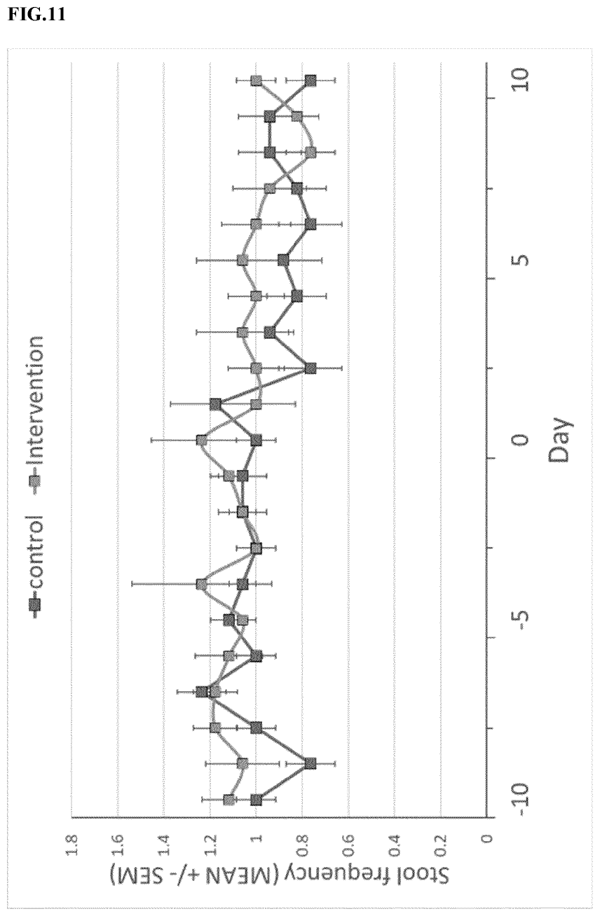

FIG. 10 is a graph showing the differences in metabolic composition with respect to a baseline. The intervention decreases the alterations at metabolic level produced by the high fat challenge. This effect is especially more significant 2 days after the high fat challenge has finished.

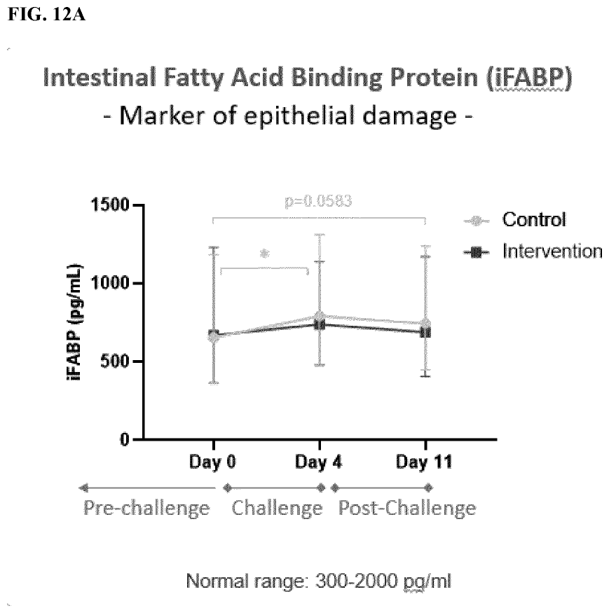

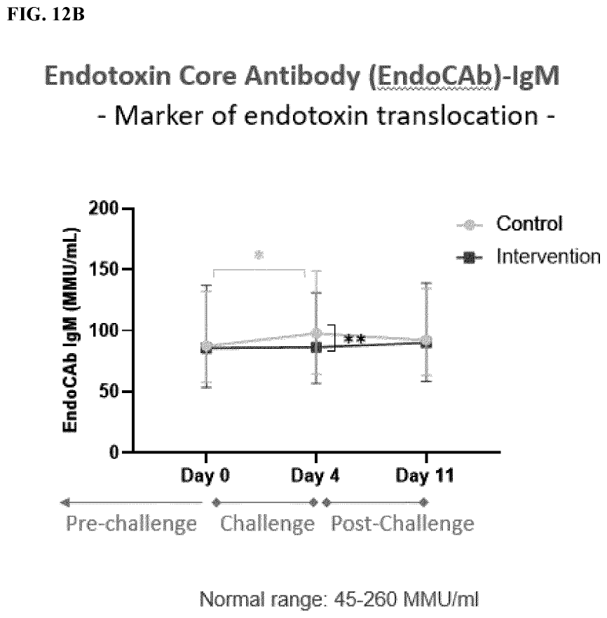

FIGS. 11, 12A and 12B show effects on the host in the clinical study. Specifically, FIG. 11 demonstrates the nutritional intervention normalizes stool frequency during the challenge, and FIGS. 12A and 12B demonstrate increased high fat diet-induced antibodies against circulating endotoxin levels are prevented by the nutritional intervention, which reflects improved gut barrier and prevention of endotoxemia.

This rigorous randomized controlled clinical study demonstrates that intake of a combination of a fiber blend and a probiotic mixture improve microbiome resilience, intestinal transit, and gut barrier and prevents endotoxemia in subjects undergoing a challenge of a diet with high fat and low fiber.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A method to enhance resilience of the microbiome in a subject, the method comprising administering, to the subject, a combination of at least one fiber and at least one probiotic.

2. The method of claim 1, wherein the subject is experiencing a microbiome stressor.

3. The method of claim 2, wherein the stressor is selected from the group consisting of a dietary stressor; antibiotic; other medications; infections; intense exercise; stress; alcohol; travel; parenteral feeding; enteral feeding; short bowel syndrome; gut inflammation; chemotherapy; colon cancer; diarrhea; proton pump inhibitors; gluten-free diet; diet free of fermentable oligo-, di-, mono-saccharides and polyols (FODMAPs); and combinations thereof.

4. A method of achieving at least one result selected from the group consisting of (i) prevention or attenuation of perturbation of microbiota; (ii) recovery after perturbation of the microbiota; and (iii) normalization of one or more of stool frequency, intestinal transit, constipation, gut permeability, endotoxemia and gut barrier function, the method comprising enhancing resilience of the microbiome in a subject in need of same by administering, to the subject, a combination of at least one fiber and at least one probiotic.

5. A method of improving gastrointestinal health, the method comprising enhancing resilience of the microbiome in a subject by administering, to the subject, a combination of at least one fiber and at least one probiotic.

6. The method of claim 1, wherein the at least one fiber is selected from the group consisting of insoluble fiber, soluble fiber, and a mixture thereof.

7. The method of claim 4, wherein the at least one fiber is selected from the group consisting of insoluble fiber, soluble fiber, and a mixture thereof.

8. The method of claim 1, wherein the at least one fiber comprises at least two fibers.

9. The method of claim 1, wherein the at least one probiotic is selected from the group consisting of Lactobacillus acidophilus, Bifidobacterium lactis, Lactobacillus rhamnosus, Bifidobacterium longum, Lactobacillus plantarum, Bifidobacterium bifidum, Lactobacillus gasseri, and mixtures thereof.

10. The method of claim 4, wherein the at least one probiotic is selected from the group consisting of Lactobacillus acidophilus, Bifidobacterium lactis, Lactobacillus rhamnosus, Bifidobacterium longum, Lactobacillus plantarum, Bifidobacterium bifidum, Lactobacillus gasseri, and mixtures thereof.

11. The method of claim 1, wherein the at least one probiotic comprises at least two probiotic strains.

12. The method of claim 1, wherein the combination of at least one fiber and at least one probiotic is orally administered to the subject.

13. The method of claim 1, wherein the subject is selected from the group consisting of a human infant, a human child, a human adolescent, a human adult, an elderly human, and an animal such as a companion animal.

14-15. (canceled)

16. The method of claim 5, wherein the at least one fiber is selected from the group consisting of insoluble fiber, soluble fiber, and a mixture thereof.

17. The method of claim 5, wherein the at least one probiotic is selected from the group consisting of Lactobacillus acidophilus, Bifidobacterium lactis, Lactobacillus rhamnosus, Bifidobacterium longum, Lactobacillus plantarum, Bifidobacterium bifidum, Lactobacillus gasseri, and mixtures thereof.
